(19) United States
(12) Patent Application Publication
(10) Pub. No.: US 2022/0392623 A1
(43) Pub. Date: Dec. 8, 2022

(54) MEDICAL DEVICE DIGITAL TWIN FOR SAFE REMOTE OPERATION

(71) Applicant: KONINKLIJKE PHILIPS N.V., EINDHOVEN (NL)

(72) Inventors: Thomas Erik AMTHOR, HAMBURG (DE); Joachim Dieter SCHMIDT, HAMBURG (DE)

(21) Appl. No.: 17/774,515

(22) PCT Filed: Nov. 11, 2020
(86) PCT No.: PCT/EP2020/081789

§ 371 Date: May 5, 2022

Related U.S. Application Data
(60) Provisional application No. 62/934,027, filed on Nov. 12, 2019.

(51) Int. Cl. G16H 40/40; G16H 40/67

(52) U.S. Cl. CPC G16H 40/40; G16H 40/67

(57) ABSTRACT

A control system (10) for controlling a medical imaging device (12) includes: an electronic controller (18) including controller memory (26) and a controller display (24). The electronic controller is operatively connected to control the medical imaging device and programmed to: store a controller state (30) in the controller memory; execute imaging device setup software (38) to cause the electronic controller to provide a setup user interface (28) on the electronic controller via which a user configures the controller state stored in the controller memory; and execute control software (40) to cause the electronic controller to control the medical imaging device to acquire images (36) in accordance with the controller state stored in the controller memory. A remote computer (18′) includes remote computer memory (26′) and a remote computer display (24′). The remote computer not operatively connected to control the medical imaging device and programmed to: receive the controller state from the electronic controller via an electronic network (46) and store the received controller state in the remote computer memory, execute the imaging device setup software to cause the remote computer to provide the setup user interface on the remote computer via which a remote user configures the controller state stored in the remote computer memory thereby creating controller state modifications (30′), and transfer the controller state modifications to the electronic controller. The electronic controller is further programmed to update the controller state stored in the controller memory with the controller state modifications.

### FIELD

The following relates generally to the imaging arts, imaging device setting modification arts, remote imaging monitoring arts, and related arts.

### BACKGROUND

The advantages of having a local technologist assisted by a more experience remote technologist or radiologist has been recognized. A suitable approach uses a screen sharing application in which the imaging device controller's display is mirrored at a remote display, along with a video camera installed in the imaging bay containing the medical imaging device, which provides a remote video feed showing the patient and capturing patient/operator interactions, and a telephone link or other natural language communication link via which a remote “operator” can advise the local operator.

Due to safety considerations, the mirrored remote display is “read-only” during clinical imaging, and the remote “operator” is unable to actually control the medical imaging device. Rather, the remote “operator” provides advice via the telephone link to a local operator, who then actually operates the medical imaging device. Consequently, the remote assistance process is cumbersome, as the remote party must make suggestions to the local technologist via telephone, instant messaging, or the like, and the suggestions are actually input to the imaging device controller by the local technologist.

The following discloses certain improvements to overcome these problems and others.

### SUMMARY

In one aspect, a control system for controlling a medical imaging device includes: an electronic controller including controller memory and a controller display. The electronic controller is operatively connected to control the medical imaging device and programmed to: store a controller state in the controller memory; execute imaging device setup software to cause the electronic controller to provide a setup user interface on the electronic controller via which a user configures the controller state stored in the controller memory; and execute control software to cause the electronic controller to control the medical imaging device to acquire images in accordance with the controller state stored in the controller memory. A remote computer includes remote computer memory and a remote computer display. The remote computer not operatively connected to control the medical imaging device and programmed to: receive the controller state from the electronic controller via an electronic network and store the received controller state in the remote computer memory, execute the imaging device setup software to cause the remote computer to provide the setup user interface on the remote computer via which a remote user configures the controller state stored in the remote computer memory thereby creating controller state modifications, and transfer the controller state modifications to the electronic controller. The electronic controller is further programmed to update the controller state stored in the controller memory with the controller state modifications.

In another aspect, a non-transitory storage medium stores instructions readable and executable by a remote computer including remote computer memory and a remote computer display to perform a remote assistance method for assisting a user of an electronic controller connected to control a medical imaging device wherein the electronic controller is programmed to execute imaging device setup software. The method includes: receiving a controller state of the electronic controller via an electronic network and storing the received controller state in the remote computer memory; executing a copy of the imaging device setup software of the electronic controller to cause the remote computer to provide a setup user interface on the remote computer via which a remote user configures the controller state stored in the remote computer memory thereby creating controller state modifications, and transferring the controller state modifications from the remote computer to the electronic controller.

In another aspect, a control method for controlling a medical imaging device using an electronic controller includes: storing a controller state of the medical imaging device in a controller memory of the electronic controller; controlling the medical imaging device using the electronic controller to acquire images accordance with the controller state stored in the controller memory; providing a setup user interface on the electronic controller via which a user configures the controller state stored in the controller memory, wherein the setup user interface includes a user input operable by the user to obtain remote setup assistance by operations performed by the electronic controller including: transferring the controller state to a remote computer via an electronic network; after the transferring, receiving controller state modifications from the remote computer via the electronic network; and updating the controller state stored in the controller memory with the controller state modifications.

One advantage resides in providing an ability to remotely modify one or more settings of an imaging device, without introducing patient safety risks associated with remote control of a medical imaging device.

Another advantage resides in providing a virtual twin of an imaging device that is remote from the imaging device that provides for remote modification of one or more settings of the imaging device.

Another advantage resides in providing a virtual twin of an imaging device that allow modifications to settings of the imaging device without actually implementing the modifications.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative control system for controlling an imaging device for in accordance with the present disclosure.

FIGS. 2 and 3 show example flow chart operations performed by the control system of FIG. 1.

FIG. 4 shows an example of an example setting modification generated by the control system of FIG. 1.

### DETAILED DESCRIPTION

In some embodiments disclosed herein, a twin of the imaging device controller is provided at the remote computer. The twin comprises a remote computer, or a virtual machine (VM) installed on a remote computer, which runs the same operating system (OS) and software as that running on the imaging device controller. The remote computer or VM also has loaded therein an image copy of all data currently stored in the memory of the imaging device controller. This includes the current hardware configuration files, current software-adjustable hardware settings, current imaging sequence settings, and any acquired images currently stored at the controller.

The twin running on the remote computer or VM is typically not intended to allow for remote control of the imaging device; accordingly, the twin does not need to include simulation of input/output interactions with the hardware of the imaging device. However, the hardware configuration files and software-adjustable hardware settings are transferred to the twin because the available hardware and their settings can impact permissible ranges of values for the imaging sequence settings. Providing any acquired images at the twin also allows the remote party to review the images to assess image quality, including performing any type of image processing that could be performed at the imaging device controller.

Hence, the remote party can interact with the twin as if interacting with the actual imaging device controller. This allows the remote party to efficiently adjust combinations of imaging sequence settings and/or software-adjustable hardware settings based on the knowledge and experience of the remote party. As the remote party is typically an experienced imaging technician or other domain expert having strong familiarity with the make/model of the imaging device, the twin provides the remote party with (a copy of) the actual imaging device controller user interface, with which the remote party is already comfortable.

To address safety concerns with remote operation, the remote party cannot actually apply any changes made to the twin running on the remote computer or VM to the actual imaging device controller. Rather, the twin maintains a record of all adjustments (e.g. hardware setting adjustments, imaging sequence setting changes) applied to the twin by the remote party. When the remote party is satisfied with the adjustments, the remote party pushes the adjustments to the imaging device controller. At the controller, these adjustments to the twin are presented to the local technician as proposed changes. The local technician can then accept or reject each proposed adjustment (or, in a variant approach, can accept or reject groups of related adjustments, say to a specific imaging sequence, or can accept or reject all adjustments) and the accepted adjustments are applied to the imaging device controller. An indication of any rejection is preferably sent back to the twin and the rejected adjustment(s) is/are rolled back at the remote computer or VM.

If further remote assistance is desired (e.g., if the initial set of adjustments does not solve the problem that led the local technician to seek the assistance of the remote party), then the twin running on the remote computer or VM is updated to the current state of the controller (e.g., memory contents copied to the twin along with any additional adjustments made by the local technician beyond those proposed adjustments that were accepted) and the process is repeated.

Because the twin is not performing remote control of the imaging device, the twin is not performing screen-sharing (read-only or otherwise) of the controller display at the twin. Likewise, the display of the twin is not shared at the local controller.

In some embodiments disclosed herein, read-only screen sharing of the display of the twin is provided at the local controller, e.g. in a separate window shown on the controller display using a conventional screen-sharing application. This could be educational for the local technician who can thereby follow the (proposed) adjustments step-by-step as they are performed by the remote party. The local technician might also provide real-time feedback via telephone or instant messaging based on the shared screen (for example, “I already tried that adjustment and it did not improve the image.”). However, any such screen sharing of the twin display at the controller would be read-only and would not actually change any settings of the imaging device controller. In similar fashion, it is contemplated to provide screen-sharing of the display of the local controller at the remote service center that hosts the remote party. This could be useful to allow the remote party to observe the local technician's manipulation of the local controller in real time. Again, however, any such screen sharing of the local controller at the remote service center would be read-only and additional to the display of the twin presented to the remote party.

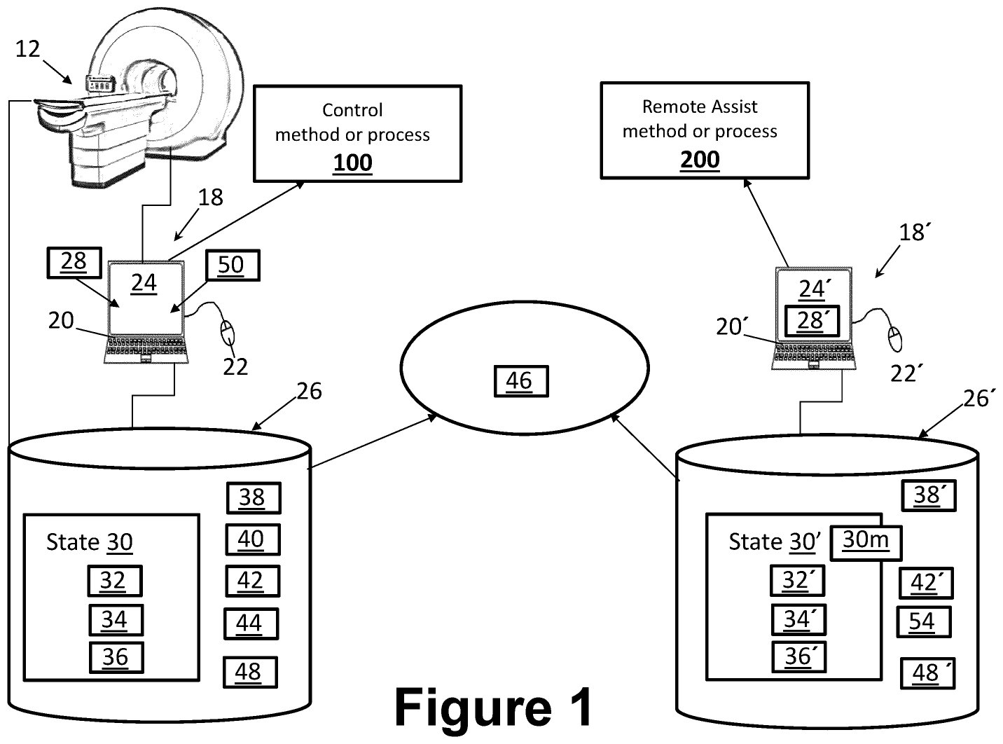

With reference to FIG. 1, an illustrative control system 10 for controlling a medical imaging device (also referred to as an image acquisition device, imaging device, and so forth) 12 shown. The image acquisition device 12 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The imaging device 12 may also be a hybrid imaging device such as a PET/CT or SPECT/CT imaging system. While a single image acquisition device 12 is shown by way of illustration in FIG. 1, more typically a medical imaging laboratory will have multiple image acquisition devices, which may be of the same and/or different imaging modalities. For example, if a hospital does many CT imaging examinations and relatively fewer MRI examinations and still fewer PET examinations, then the hospital's imaging laboratory (sometimes called the “radiology lab” or some other similar nomenclature) may have three CT scanners, two MRI scanners, and only a single PET scanner. Accordingly, each image acquisition device 12 would include a corresponding control system 10. This is merely an example.

FIG. 1 also shows an electronic controller 18, such as an electronic processing device, a workstation computer, or more generally a computer, operatively connected to control the medical imaging device 12. Alternatively, the electronic processing device 18 can be embodied as a server computer or a plurality of server computers, e.g. interconnected to form a server cluster, cloud computing resource, or so forth. The electronic controller 18 includes typical components, such as an electronic processor 20 (e.g., a microprocessor), at least one user input device (e.g., a mouse, a keyboard, a trackball, and/or the like) 22, and a controller display device 24 (e.g. an LCD display, plasma display, cathode ray tube display, and/or so forth). In some embodiments, the display device 24 can be a separate component from the workstation 18. The electronic processor 20 is operatively connected with a controller memory 26 embodied as one or more non-transitory storage media. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a setup graphical user interface (GUI) 28 for display on the controller display device 24.

The controller memory 26 is programmed to store hardware and software related to operation of the imaging device 12. For example, the controller memory 26 is programmed to store a controller state 30 of the imaging device 12. The controller state 30 can include operational settings of the imaging device 12. The controller settings 32 can include image acquisition settings 32. In another example, the controller state 30 includes a device hardware configuration 34 for the medical imaging device 12. In a further example, the controller state 30 includes images 36 stored in the controller memory 26.

The controller memory 26 stores imaging device setup software 38 that allows a user (e.g., a radiologist, etc.) to configure the settings 32 of the controller state 30. The controller memory 26 also stores control software 40 that causes the electronic controller 18 to control the medical imaging device 12 to acquire the images 36 of a patient in accordance with the stored controller state 30. Usually, the controller memory 26 further stores image presentation software 42 to cause the electronic controller 18 to display and manipulate the images 36 on the controller display device 24. Furthermore, as disclosed herein, the controller memory 26 stores remote update software 44 that causes the electronic controller 18 to update the controller state 30 based on feedback received from the twin, as will be described herein. In addition, the electronic controller 18 is programmed to run an operating system (OS) 48 under which the imaging device setup software 38, the control software 40, the image presentation software 42, and the remote update software 44 are executed.

The electronic controller 18 is configured as described above to perform a control method or process 100 for controlling the medical imaging device 12 via the control software 40 to acquire medical images of patients in accordance with the settings configured via the setup software 38, and for presenting the acquired images for review by the technician via the presentation software 42, and as disclosed herein for receiving remote updates from the twin via the remote update software 44. The non-transitory storage medium 26 stores the instructions 38, 40, 42, 44 which are readable and executable by the at least one electronic processor 20 of the electronic controller 18 to perform disclosed operations including performing the control method or process 100. In some examples, the method 100 may be performed at least in part by cloud processing.

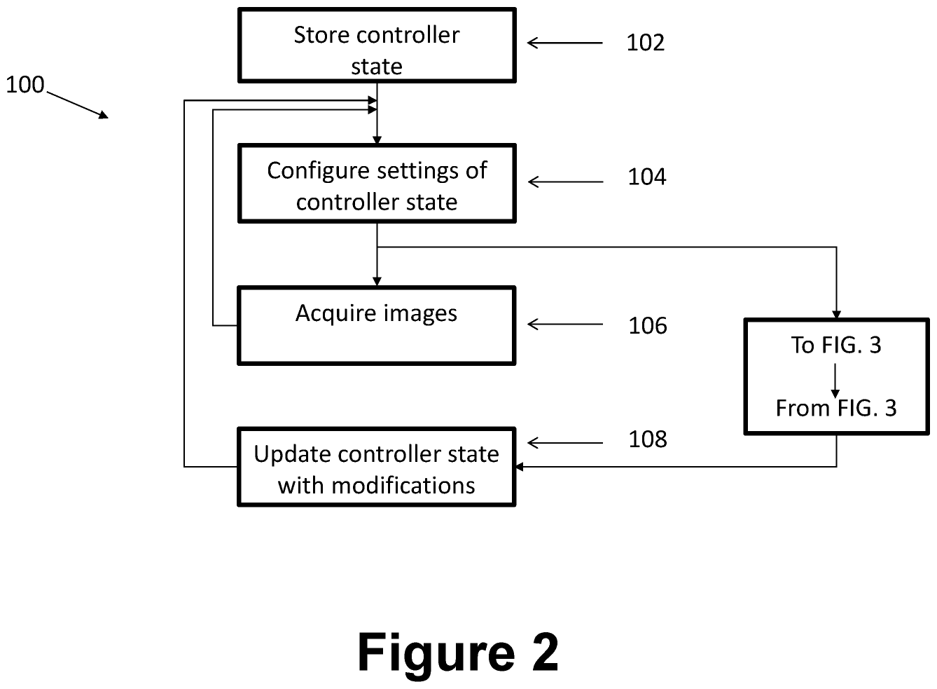

With reference to FIG. 2, and with continuing reference to FIG. 1, an illustrative embodiment of control method 100 is diagrammatically shown as a flowchart. At an operation 102, the electronic controller 18 is programmed to store the controller state 30 of the imaging device 12 (including the image acquisition settings 32, the device hardware configuration 34, and/or the images 36) in the controller memory 26.

At an operation 104, the electronic controller 18 is programmed to execute the imaging device setup software 38 to cause the electronic controller to provide the setup user interface 28 on the GUI to allow a user to configure the settings 32 of the controller state 30. The setup user interface 28 is configured in accordance with the device hardware configuration 34 of the controller state 30.

At an operation 106, the electronic controller 18 is programmed to execute the control software 40 to cause the electronic controller to control the medical imaging device 12 to acquire the images 36 in accordance with the stored controller state 30. The images 36 can be stored in the controller memory 26. Additionally or alternatively, the electronic controller 18 is programmed to execute the image presentation software 42 to display and manipulate the images 36 on the display device 24. The manipulated images 36 can also be stored in the controller memory 26. The process 100 may stop at 106 if the local technician is satisfied with the images and the imaging examination is complete; otherwise, the process passes back to operation 104 where the local operator adjusts the settings to improve the images, or moves on to another set up another imaging sequence to be performed in the imaging examination.

Referring back to FIG. 1, the control system 10 further includes a remote computer 18′ on which a twin of the electronic controller 18 is implemented. The twin may be implemented directly on the computer 18′, or may be implemented on a virtual machine (VM) running on the computer 18′. As used herein, features of the remote computer 18′ similar to those of the electronic controller 18 have a common reference number followed by a “prime” symbol.

The remote computer 18′ is configured as an electronic processing device, a workstation computer, or more generally a computer, and is not operatively connected to control the medical imaging device 12. Alternatively, the remote computer 18′ can be embodied as a server computer or a plurality of server computers, e.g. interconnected to form a server cluster, cloud computing resource, or so forth. The electronic controller 18 includes typical components, such as an electronic processor 20′, at least one user input device 22′, and a remote computer display device 24′. In some embodiments, the display device 24′ can be a separate component from the remote computer 18′. The electronic processor 20′ is operatively connected with a remote computer memory 26′ embodied as one or more non-transitory storage media. The remote computer non-transitory storage media 26′ stores instructions executable by the at least one electronic processor 20′. The instructions include instructions to generate a setup GUI 28′ for display on the remote computer display device 24′.

The remote computer 18 implements the twin directly on the remote computer 18′, or in a VM running on the computer 18′. Direct implementation may be feasible if the remote computer 18′ is running the same operating system as the electronic controller 18. However, implementation of the twin in a VM running on the remote computer 18′ has a number of advantages. It permits the remote computer 18′ to be running a different operating system (OS) than the OS 48 of the electronic controller 18. Furthermore, using a VM places the twin in a sandbox, meaning that the twin running in a VM does not impact other processes that may optionally be running on the remote computer 18′. Such “other processes” may, for example, include an electronic mail program, word processor, or other programs that may be used by the remote party, and/or may include video streaming software for receiving and presenting video of the imaging device 12 acquired by a video camera (not shown) in the imaging bay, and/or may include screen sharing software providing the remote party with a real-time view of content shown on the display 24 of the electronic controller 18. Yet a further advantage to using a VM is that if the remote party is providing assistance to multiple different local technicians in different imaging bays, then the remote computer 18′ can run multiple twins (one for each of the different imaging bays) in separate VMs, and again the use of VMs ensures that the twins do not interfere with each other. Some suitable VM environments are available, for example, from VMware, Inc. (Palo Alto, Calif., USA) or Parallels Inc. (Bellevue, Wash., USA), by way of non-limiting illustrative example.

The remote computer memory 26′ also stores a copy 30′ of the controller state 30, (including a copy 32′ of the controller settings 32, a copy 34′ of the device hardware configuration 34, and a copy 36′ of the images 36), a copy 38′ of the imaging device setup software 38, a copy 42′ of the image presentation software 42, and a copy 48′ (i.e. second instance 48′) of the OS 48. Typically, the remote computer memory 26′ does not store a copy of the control software 40, since the remote computer 18′ is not intended to control the imaging device 12. However, in some specific implementations the imaging device setup software 38 and the control software 40 may be integral with one another, i.e. written as a single piece of software, or the two pieces of software 38, 40 may only available and installable as a combined package. In this case, a copy of the combined software 38, 40 is loaded onto the remote computer 18′ (or the VM running on same) in order to provide the copy 38′ of the device hardware configuration 34, and the control imaging device control aspects of this combined software package will be inoperable. The OS 48 runs on the remote computer 18′ or on a VM executing on the remote computer 18′, and allows the user to execute (the copy of) the image device setup software 38′ under the (copy or second instance of) OS 48′ running on the remote computer 18′ or VM, and allows the user to execute (the copy of) the image presentation software 42′ under the (copy or second instance of) OS 48′ running on the remote computer 18′ or VM.

Furthermore, the remote computer memory 26′ stores modification tracking software 54 which, when executed by the remote computer 18′ or VM running on same operates to track controller state modifications made on the twin and ultimately to push the modifications to the electronic controller 18. The modification tracking software 54 may execute as a background process that monitors and tracks changes made to (the copy of) the controller state 30′. Alternatively, if the imaging device setup software 38′ has “undo” capability then the imaging device setup software 38′ will be logging modifications automatically (in order to enable undoing them if the operator so requests), and in this case the modification tracking software 54 may operate by referencing the “undo” stack of modifications automatically maintained to facilitate the “undo” capability. In order to receive the controller state 30 from the electronic controller 18 and to push the state modifications made on the twin to the electronic controller 18, the remote computer 18′ is operatively connected to the electronic controller 18 via an electronic network 46 (e.g., Internet, Wi-Fi, a personal area network (PAN), a virtual private network (VPN), and so forth). Optionally, the electronic network 46 may provide other connectivity between the imaging bay of the imaging device 12 and the remote servicing center hosting the remote computer 18′, such as video streaming capability for streaming video of the imaging device 12 to the remote computer 18′, transferring screen sharing data (this screen sharing again would be separate from operation of the twin described herein), and providing a natural language link such as a video chat or voice over Internet protocol (VOIP) telephonic link via which the local technician and the remote operator can converse.

The remote computer 18′ is configured as described above to perform a remote assistance method or process 200 for assisting a user of the electronic controller 18 connected to control the medical imaging device 12. The non-transitory storage medium 26′ stores instructions which are readable and executable by the at least one electronic processor 20′ of the remote computer 18′ to perform disclosed operations including performing the remote assistance method or process 200. In some examples, the method 200 may be performed at least in part by cloud processing.

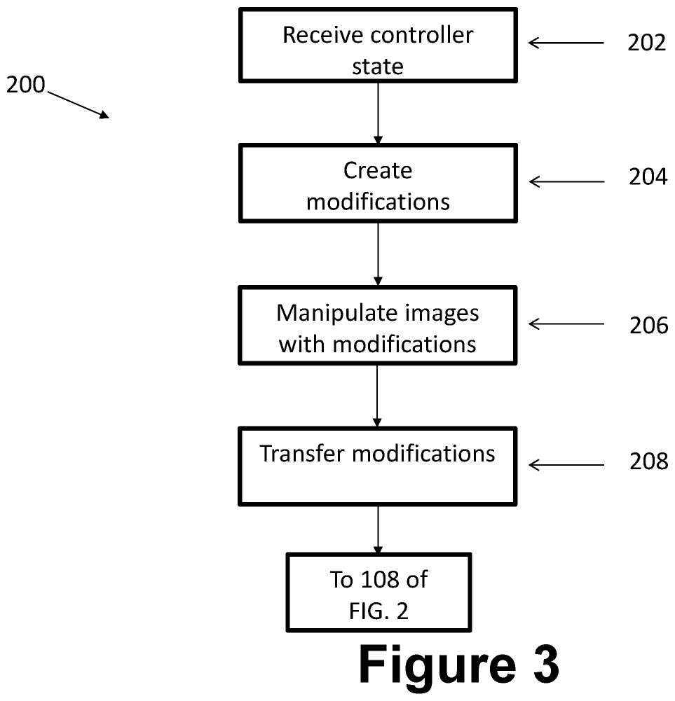

With reference to FIG. 3, and with continuing reference to FIG. 1, an illustrative embodiment of remote assistance method 200 is diagrammatically shown as a flowchart. At an operation 202, the remote computer 18′ is programmed to receive the controller state 30 (including the image acquisition settings 32, the device hardware configuration 34, and/or the images 36) from the electronic controller 18 via the electronic network 46. The controller state 30 is stored as copy 30′ in the remote computer memory 26.

At an operation 204, the remote computer 18′ is programmed to execute the imaging device setup software 38′ to provide the setup user interface 28′ on the remote computer display device 24′ via which a remote user (e.g., a technologist) configures the received controller state 30′ to create controller state modifications 30m. The setup user interface 28′ is configured in accordance with the device hardware configuration 34′. The controller state modifications 30m typically include modifications to the image acquisition settings 32′.

At an operation 206, the remote computer 18′ is programmed to execute the image presentation software 42′ to display and manipulate the images 36 stored in the remote computer memory 26′ on the remote computer display device 24′. The manipulated images 36 can also be stored in the remote computer memory 26′ and can constitute controller state modifications 30m. (Depending upon the nature of the assistance being provided, the remote party may omit the operation 206, i.e. may not load and use the image presentation software 42′).

At an operation 208, the remote computer 18′ is programmed (e.g., by the modification tracking software 54) to transfer the controller state modifications 30m to the electronic controller 18. At this point, the control method 100 resumes. It will be appreciated that the transferred control state modifications 30m are not automatically implemented into the controller state 30 without authorization from the radiologist at the imaging device 12. In other words, the remote computer 18′ cannot control the imaging device 12.

Rather, referring back to FIG. 2, the control method 100 resumes at an operation 108 when the electronic controller 18 receives the controller state modifications 30′ from the remote computer 18′. At the operation 108, the electronic controller 18 is programmed to update the controller state 30 with the controller state modifications 30m. To do so, the remote update software 44 is executed to cause the electronic controller 18 to display a remote update GUI 50 on the controller display device 24. The remote update GUI 50 displays the controller state modifications 30m on the controller display device 24. The local technologist can use the at least one input device 22 to input a user input selecting an acceptance or rejection of the controller state modifications 30m. If the local technologist accepts the controller state modifications 30m, the controller state 30 stored in the controller memory 26 is updated with the controller state modifications. If, however, the local technologist rejects the controller state modifications 30m, then a message is sent back to the remote computer 18′ that is displayed on the remote computer display device 24′ indicating that the modifications were not accepted by the local technologist. In this case, the changes may optionally be rolled back in (the copy of) the state 30′ at the remote computer 18′. If these modifications do not address the problem being dealt with by the local technologist, then the operations 204 and/or 206 can be repeated to generate a new set of modifications, which are then transmitted to the electronic controller 18.

In some examples of some suitable implementations of the update operation 108 and the update GUI 50, the controller state modifications 30m are displayed as a list on the controller display device 24 in which the radiologist can accept or reject individual ones of the controller state modifications (e.g., via the at least one user input device 22). For those controller state modifications 30m that are accepted, the accepted modifications are implemented into the controller state 30. For the rejected controller state modifications 30m, a message is sent to the technologist that is displayed on the remote computer display device 24′ indicating which modifications were not accepted by the local technologist, and the rejected modifications are optionally rolled back at the remote computer 18′. The operations 204 and/or 206 can be repeated to generate a new set of modifications, which are then transmitted to the electronic controller 18, until the local technologist accepts all of the modifications.

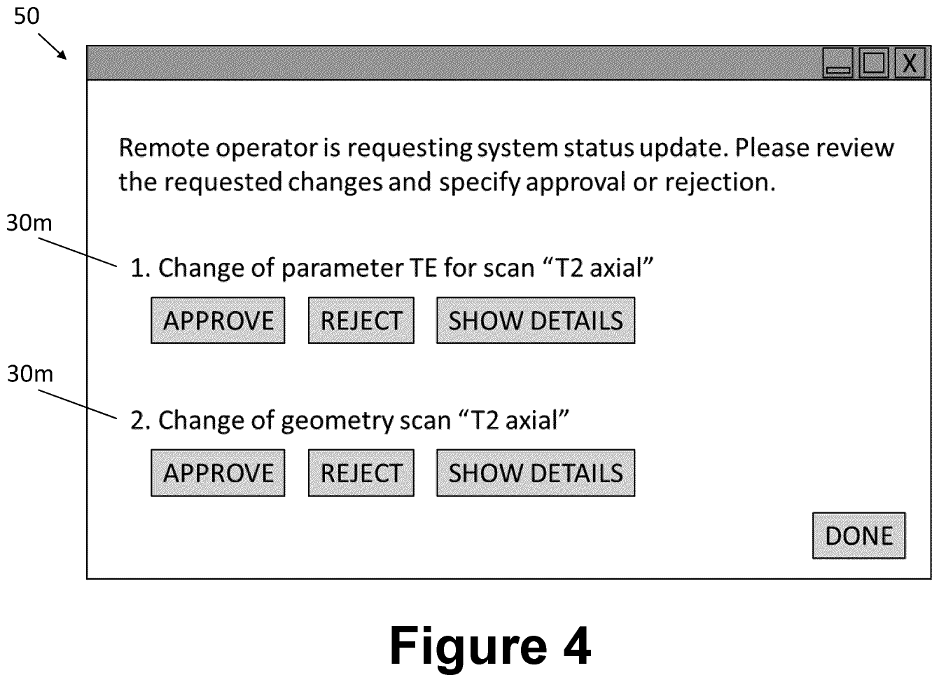

FIG. 4 shows an example of the remote update GUI 50 for display on the controller display device 24. As shown in FIG. 4, the GUI 50 shows two controller state modifications 30m—a change or parameter modification and a geometry scan modification. Each modification 30′ includes an “Accept” and “Reject” option for the radiologist to select. In addition, a “Show Details” option can be selected, which would open a window explaining the reason for the modification 30′ in more detail. An indication of the accept or reject options can be sent to the remote computer 18′ for the technologist to view. The illustrative example of FIG. 4 is directed to an embodiment in which the imaging device 12 is an MRI device; however, as previously noted the disclosed approaches employing a twin to facilitate assistance from a remote party can be readily applied for other imaging modalities.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A control system for controlling a medical imaging device, the control system comprising:
an electronic controller including controller memory and a controller display, the electronic controller operatively connected to control the medical imaging device and programmed to:
store a controller state in the controller memory;
execute imaging device setup software to cause the electronic controller to provide a setup user interface on the electronic controller via which a user configures the controller state stored in the controller memory; and
execute control software to cause the electronic controller to control the medical imaging device to acquire images in accordance with the controller state stored in the controller memory; and
a remote computer including remote computer memory and a remote computer display, the remote computer not operatively connected to control the medical imaging device, the remote computer programmed to:
receive the controller state from the electronic controller via an electronic network and store the received controller state in the remote computer memory,
execute the imaging device setup software to cause the remote computer to provide the setup user interface on the remote computer via which a remote user configures the controller state stored in the remote computer memory thereby creating controller state modifications, and
transfer the controller state modifications to the electronic controller;
wherein the electronic controller is further programmed to update the controller state stored in the controller memory with the controller state modifications.

2. The control system of claim 1, wherein:
the controller state includes image acquisition settings, and the controller state modifications include image acquisition settings modifications.

3. The control system of claim 2, wherein:
the controller state further includes a device hardware configuration for the medical imaging device;
the setup user interface provided on the electronic controller is configured in accordance with the device hardware configuration; and
the setup user interface provided on the remote computer is configured in accordance with the device hardware configuration.

4. The control system of claim 1, wherein:
the controller state includes images stored in the controller memory;
the electronic controller is further programmed to execute image presentation software to cause the electronic controller to display and manipulate the images stored in the controller memory;
the controller state received at the remote computer and stored in the remote computer memory includes the images stored in the controller memory, and
the remote computer is further programmed to execute the image presentation software to cause the remote computer to display and manipulate the images stored in the remote computer memory.

5. The control system of claim 1, wherein the electronic controller is programmed to run an operating system under which the imaging device setup software and the control software are executed, and the remote computer is programmed to execute the imaging device setup software under the OS running in a virtual machine on the remote computer.

6. The control system of claim 1, wherein the electronic controller is programmed to update the controller state stored in the controller memory with the controller state modifications by executing remote update software to cause the electronic controller to:
display a remote update user interface on the electronic controller which presents the controller state modifications on the controller display as one or more proposed controller state modifications and via which the user selects to accept or reject each proposed controller state modification; and
update the controller state with only those controller state modifications that are accepted by the user.

7. A non-transitory storage medium storing instructions readable and executable by a remote computer including remote computer memory and a remote computer display to perform a remote assistance method for assisting a user of an electronic controller connected to control a medical imaging device wherein the electronic controller is programmed to execute imaging device setup software, the method including:
receiving a controller state of the electronic controller via an electronic network and storing the received controller state in the remote computer memory,
executing a copy of the imaging device setup software of the electronic controller to cause the remote computer to provide a setup user interface on the remote computer via which a remote user configures the controller state stored in the remote computer memory thereby creating controller state modifications, and
transferring the controller state modifications from the remote computer to the electronic controller.

8. The non-transitory storage medium of claim 7, wherein:
the controller state received from the electronic controller includes image acquisition settings, and the controller state modifications include image acquisition settings modifications.

9. The non-transitory storage medium of claim 8, wherein:
the controller state received from the electronic controller further includes a device hardware configuration for the medical imaging device; and
the setup user interface provided on the remote computer is configured in accordance with the device hardware configuration.

10. The non-transitory storage medium of claim 8, wherein the electronic controller is further programmed to execute image presentation software; and wherein:
the controller state received from the electronic controller includes images stored in the controller memory,
the electronic controller is further programmed to execute a copy of the image presentation software of the electronic controller to cause the remote computer to display and manipulate the images stored in the remote computer memory.

11. The non-transitory storage medium of claim 7, wherein instructions readable and executable by the remote computer do not include instructions readable and executable by the remote computer to control the medical imaging device.

12. A control method for controlling a medical imaging device using an electronic controller, the control method comprising:
storing a controller state of the medical imaging device in a controller memory of the electronic controller;
controlling the medical imaging device using the electronic controller to acquire images in accordance with the controller state stored in the controller memory;
providing a setup user interface on the electronic controller via which a user configures the controller state stored in the controller memory, wherein the setup user interface includes a user input operable by the user to obtain remote setup assistance by operations performed by the electronic controller including:
transferring the controller state to a remote computer via an electronic network;
after the transferring, receiving controller state modifications from the remote computer via the electronic network; and
updating the controller state stored in the controller memory with the controller state modifications.

13. The control method of claim 12, wherein the updating includes:
presenting the controller state modifications on the controller display as one or more proposed controller state modifications;
receiving, from a user via a user input device of the electronic controller, acceptance or rejection of each proposed controller state modification; and
updating the controller state with only those controller state modifications that receive an acceptance from the user.

14. The control method of claim 12, wherein the controller state includes image acquisition settings, the transferring of the controller state includes transferring the image acquisition settings to the remote computer, and the received controller state modifications include image acquisition settings modifications.

15. The control method of claim 14, wherein:
the controller state includes a device hardware configuration for the medical imaging device;
the setup user interface provided on the electronic controller is configured in accordance with the device hardware configuration, and
the transferring of the controller state includes transferring the device hardware configuration for the medical imaging device to the remote computer.

16. The control method of claim 12, wherein the controller state includes images stored in the controller memory, and the transferring of the controller state includes transferring the images stored in the controller memory to the remote computer.

17. The control method of claim 12, wherein the control method does not include controlling the medical imaging device using the remote computer.

18. The control method of claim 12, further comprising:
storing the controller state received at the remote computer from the electronic controller in a remote computer memory of the remote computer;
providing the setup user interface on the remote computer via which a remote user configures the controller state stored in the remote computer memory thereby generating the controller state modifications; and
transferring the controller state modifications to the electronic controller via the electronic network.

19. The control method of claim 18, wherein:
the controller state received from the electronic controller includes image acquisition settings, and the controller state modifications include image acquisition settings modifications;
the controller state received from the electronic controller further includes a device hardware configuration for the medical imaging device; and
the setup user interface provided on the remote computer is configured in accordance with the device hardware configuration.

20. The control method of claim 18, wherein the electronic controller is further programmed to execute image presentation software; and wherein:
the controller state received from the electronic controller includes images stored in the controller memory,
the electronic controller is further programmed to execute a copy of the image presentation software of the electronic controller to cause the remote computer to display and manipulate the images stored in the remote computer memory.
